(19) United States
(12) Patent Application Publication
(10) Pub. No.: US 2024/0150852 A1
(43) Pub. Date: May 9, 2024

(54) Graphene-based malaria sensor, methods and uses thereof

(71) Applicants: International Iberian Nanotechnology Laboratory (INL), Braga (PT); Universidade do Minho, Braga (PT)

(72) Inventors: Bruno Miguel BARROSO RODRIGUES DE ALMEIDA, Braga (PT); Joana Rafaela LARA GUERREIRO, Porto (PT); Neide Marina VIEIRA PEREIRA, Braga (PT); João Pedro DOS SANTOS HALL AGORRETA ALPUIM, Braga (PT); Jérôme Gilles OLLIVIER BORME, Braga (PT); Pedro Eduardo MENDES FERREIRA, Penafiel (PT); Rui César DE ALMEIDA CAMPOS, Lamego (PT)

(21) Appl. No.: 18/548,190

(22) PCT Filed: Feb. 28, 2022
(86) PCT No.: PCT/IB22/51743

§ 371 Date: Aug. 28, 2023

(30) Foreign Application Priority Data
Feb. 26, 2021 (PT) 117090

(51) Int. Cl. C12Q 1/6893; B01L 3/00; C12Q 1/6837

(52) U.S. Cl. CPC C12Q 1/6893; B01L 3/502761; C12Q 1/6837; B01L 2200/0647; B01L 2200/16; B01L 2300/0645; C12Q 2600/156; C12Q 2600/16; C12Q 2600/166

(57) ABSTRACT

The present disclosure relates to a monolayer graphene-based multiplex malaria diagnostic sensor. Specifically, a monolayer graphene-based sensor that is able to simultaneously detect the presence of different Plasmodium species, presence of drug-resistant Plasmodium species, and also the presence of a relevant polymorphism in a subject. The present disclosure also relates to a monolayer graphene-based sensor, method and kit for a rapid diagnosis of malaria using a non-invasive biological sample obtained from a subject, preferably in saliva or urine samples.

### TECHNICAL FIELD

The present disclosure relates to a monolayer graphene-based multiplex malaria diagnostic sensor. Specifically, a monolayer graphene-based sensor that is able to simultaneously detect the presence of different Plasmodium species, presence of drug-resistant Plasmodium species, and also the presence of a relevant polymorphism in a subject, in particular G6PD single nucleotide polymorphisms.

The present disclosure also relates to a monolayer graphene-based sensor, method and kit for a rapid diagnosis of malaria using a non-invasive biological sample obtained from a subject, preferably in saliva or urine samples.

### BACKGROUND

Malaria is one of the deadliest infectious diseases in the world which can be prevented through timely diagnosis and treatment. However, current malaria diagnostic tools have limitations. Existent RDTs for malaria are able to detect one species (P. falciparum) or multiple species (P. vivax, P. malariae, P. ovale) but require human interpretation and make use of blood invasive samples, due to its high concentration of parasites. Additionally, prevalence of parasites resistant to artemisinin and other drugs used to treat malaria, is rising at an alarming rate, compromising the treatment. Moreover, millions of people in endemic regions have gene mutations (G6PD) which confers a potential risk of hemolysis by the commonly prescribed antimalarial drugs. Screening of these types of mutations can prevent unnecessary deaths. Therefore, novel diagnostic tools for malaria are urgently needed. The use of a monolayer graphene-based multiplex malaria diagnostic sensor with ability to detect malaria spp, drug resistance and host mutations is thus very beneficial. The test result will make it possible to simultaneously identify the type of malaria parasite as well as its resistance to drugs, enabling a more targeted and efficient treatment with lower risks, and uses non-invasive samples such as saliva.

Document U.S. Ser. No. 10/020,300-B2 discloses arrays may be employed to detect the presence and/or concentration changes of various analyte types in chemical and/or biological processes. Specifically, the system may comprise graphene and may detect DNA hybridization and/or sequencing reactions.

Document U.S. Ser. No. 10/793,898B2 discloses a method, systems, and nano-sensor devices for detecting or discriminating nucleic acids with a single nucleotide resolution based on nucleic acid strand displacement.

Document WO2016164783 discloses a system and method for DNA sequencing and blood chemistry analysis. Specifically, a system comprising a plurality of transistors, wherein at least one transistor comprises graphene, whereby electrical properties of the at least one transistor changes in response to contact with a DNA sequence.

Document CN107051601 discloses nucleic acid detection microfluidic chip based on graphene field effect tube. Specifically, nucleic acid detection microfluidic chip based on graphene field effect tubes.

Document JP2012247189 discloses a graphene sensor for detecting substance species. Specifically, the graphene sensor comprises a DNA fragment having a known base sequence as a functional group.

Document CN109580584 discloses a saliva diagnostic sensor comprising graphene.

These facts are disclosed in order to illustrate the technical problem addressed by the present disclosure.

### GENERAL DESCRIPTION

The present disclosure relates to a monolayer graphene-based multiplex malaria diagnostic sensor. Specifically, a monolayer graphene-based sensor that is able to simultaneously detect the presence of different Plasmodium species, presence of drug-resistant Plasmodium species, and also the presence of G6PD single nucleotide polymorphism in the test subject.

The disclosed diagnostic sensor is stable in a wide range of temperature, compatible with non-invasive sampling methods (such as saliva or urine), and returns a result rapidly, preferably in less than one hour. With the retrieved results it is possible to conclude about the presence or absence of Plasmodium species in the biological sample, and also design a suitable treatment based on drug resistance and/or polymorphisms detected.

The advantage of the sensor of the present disclosure is that it can be deployed to various settings, especially malaria rampant settings where it is more often than not impossible to set up the full spectrum of diagnostic laboratory tests required to accurately detect and diagnose malaria. Additionally, the sensor of the present disclosure is especially advantageous for settings where it will be challenging to provide refrigeration for temperature control and to provide phlebotomy expertise to obtain blood samples. Thus, the sensor of the present disclosure is heat resistant and utilizes saliva as a diagnostic sample makes it ideal for mass, rapid, field deployment.

In an embodiment, the present disclosure relates to a monolayer graphene-based sensor for a rapid diagnosis of malaria using a non-invasive biological sample obtained from a subject.

In an embodiment, the sensor comprises the following elements:
at least 3 different isolated/synthetic nucleic acid probes for identifying the presence of at least 3 different Plasmodium species in the biological sample;
a linker for binding the isolated/synthetic nucleic acid probes to the graphene sensor, wherein the linker is selected from the following list: 1-pyrenebutyric acid succinimidyl ester (PBSE), (9-Fluorenylmethoxycarbonyloxy)succinimide (Fmoc-ONSu), acridine orange succinimidyl ester (AO), or mixtures thereof;
at least 1 isolated/synthetic nucleic acid probe for identifying the presence of at least 1 Plasmodium species that is resistant to at least 1 antimalaria drug;
at least 1 isolated/synthetic nucleic acid probe for detecting the presence of at least 1 single nucleotide polymorphism in the subject that influences the malaria treatment response of the subject.

The sequences of nucleic acid probes of the present disclosure can be obtained by isolation or synthesis of deoxyribonucleic acid (DNA). Isolated DNA is a DNA that results from an extraction process in which the DNA present in the nucleus of a cell has been separated from other cellular components; DNA synthesis relates to the artificial creation of DNA, that results in synthetic DNA.

In an embodiment, the sensor may further comprise at least 1 isolated/synthetic nucleic acid probe for confirming the human origin of the biological sample (positive control).

In an embodiment, the sensor is able to detect the presence of different Plasmodium species, presence of drug-resistant Plasmodium species and the presence of G6PD single nucleotide polymorphism in a saliva sample or a urine sample.

In an embodiment, the sensor is able to detect the presence of different Plasmodium species, presence of drug-resistant Plasmodium species and the presence of G6PD single nucleotide polymorphisms in less than one hour, preferably less than 45 minutes, more preferably less than 40 minutes.

In an embodiment, the isolated/synthetic nucleic acid probes for functionalizing are selected from deoxyribonucleic acid probes, ribonucleic acid probes, locked nucleic acid probes, or mixtures thereof.

In an embodiment, the sensor comprises at least 3 different isolated/synthetic nucleic acid probes for identifying the presence of at least 3 different Plasmodium species in the biological sample and a human control, wherein the isolated/synthetic nucleic acid probes comprise at least a sequence 90% identical to the sequences of the following list, or mixtures thereof. Preferably 91% identical, 92% identical, 93% identical, 94% identical, 95% identical, 96% identical, 97% identical, 98% identical, 99% identical or identical.

| SEQ | | | |
|---|---|---|---|
| ID | | | |
| No | | | |
| 1 | P. falciparum | cytB | GTTTTAGTTATATTATCTAC |
| 2 | P. falciparum | coxI | ATATGCATATTATAGTATAC |
| 3 | P. falciparum | coxIII | CCTATAATCCTATTAATATT |
| 4 | P. falciparum | mitochondrial | GAACTCTATAAATAACCAG |
| | | | ACTATTTCAAC |
| 5 | P. falciparum | mitochondrial | CTGTAATTACTAACTTGTTA |
| | | | TCCTCTATTC |
| 6 | P. vivax | cytB | GCTATATTAGTTAATACATA |
| 7 | P. vivax | coxI | CTATATTAATATCTATACCT |
| 8 | P. vivax | coxIII | CAATATAAGATATACCATAT |
| 9 | P. vivax | mitochondrial | GTATGGATCGAATCTTACTT |
| | | | ATTCATATC |
| 10 | P. vivax | mitochondrial | TTTAGTATCTGGTATTGCTA |
| | | | GTATTATGTC |
| 11 | P. knowlesi | cytB | GTCATAACTAATTTATTATC |
| 12 | P. knowlesi | coxI | ATTCTATAATTATACTATGG |
| 13 | P. knowlesi | coxIII | GTATGAGGTAATAATATATA |
| 14 | P. knowlesi | mitochondrial | GAATATAATCACCTGTTATA |
| | | | ATGTTCTAGG |
| 15 | P. knowlesi | mitochondrial | CCTTCACTATATAATGGATA |
| | | | TGGAGATAAA |
| 16 | P. ovale | cytB | TATACATATATTCTTCTTAC |
| 17 | P. ovale | coxI | CTATATTATATCAACATCTA |
| 18 | P. ovale | coxIII | TATACCTTCATTATATAAAG |
| 19 | P. ovale | mitochondrial | CTTTCATATTAGTCATATTA |
| | | | TCTACAGCTG |
| 20 | P. ovale | mitochondrial | CCATTATAGGATTATTTACA |
| | | | ACAGTAAGTG |
| 21 | P. malariae | cytB | TAACTACTATTATACAATTC |
| 22 | P. malariae | coxI | GATTAACATTAGGTATATTA |
| 23 | P. malariae | coxIII | CCATCATTAATATAATATTC |
| 24 | P. malariae | mitochondrial | CATTAAGTACTTCTCTTATG |
| | | | TCTTTATCTC |
| 25 | P. malariae | mitochondrial | CTATGAGTTGTATAGCTATA |
| | | | TTAGGAAG |
| 26 | Plasmodium | mitochondrial | GGATAATTCTATTTATTAG |
| | spp | | GAGTCTC |
| 27 | Plasmodium | mitochondrial | AACAGGTTATAGTATATAT |
| | spp | | AGAGCTC |
| 28 | Homo sapiens | mitochondrial | GCCAACTAATATTTCACTTT |
| | | | ACATCCAAA |
| 29 | Homo sapiens | mitochondrial | GGCATTTTGTAGATGTGATT |
| | | | TGACTATT |
| 74 | Homo sapiens | cytB | CATTATTGCAGCCCTAGCAA |
| 75 | Homo sapiens | coxI | ATACCTATTATTCGGCGCAT |
| 76 | Homo sapiens | coxIII | TTCCTCACTATCTGCTTCAT |

In an embodiment, the sensor comprises at least 5 different isolated/synthetic nucleic acid probes for identifying the presence of at least 5 different Plasmodium species in the biological sample, wherein the isolated/synthetic nucleic acid probes comprise at least a sequence 90% identical to the sequences of the following list, or mixtures thereof. Preferably 91% identical, 92% identical, 93% identical, 94% identical, 95% identical, 96% identical, 97% identical, 98% identical, 99% identical or identical.

| SEQ | | | |
|---|---|---|---|
| ID | | | |
| No | | | |
| 1 | P. falciparum | cytB | GTTTTAGTTATATTATCTAC |
| 2 | P. falciparum | coxI | ATATGCATATTATAGTATAC |
| 3 | P. falciparum | coxIII | CCTATAATCCTATTAATATT |
| 4 | P. falciparum | mitochondrial | GAACTCTATAAATAACCAGA |
| | | | CTATTTCAAC |
| 5 | P. falciparum | mitochondrial | CTGTAATTACTAACTTGTTATC |
| | | | CTCTATTC |
| 6 | P. vivax | cytB | GCTATATTAGTTAATACATA |
| 7 | P. vivax | coxI | CTATATTAATATCTATACCT |
| 8 | P. vivax | coxIII | CAATATAAGATATACCATAT |
| 9 | P. vivax | mitochondrial | GTATGGATCGAATCTTACTT |
| | | | ATTCATATC |
| 10 | P. vivax | mitochondrial | TTTAGTATCTGGTATTGCTA |
| | | | GTATTATGTC |
| 11 | P. knowlesi | cytB | GTCATAACTAATTTATTATC |
| 12 | P. knowlesi | coxI | ATTCTATAATTATACTATGG |
| 13 | P. knowlesi | coxIII | GTATGAGGTAATAATATATA |
| 14 | P. knowlesi | mitochondrial | GAATATAATCACCTGTTATAA |
| | | | TGTTCTAGG |
| 15 | P. knowlesi | mitochondrial | CCTTCACTATATAATGGATAT |
| | | | GGAGATAAA |
| 16 | P. ovale | cytB | TATACATATATTCTTCTTAC |
| 17 | P. ovale | coxI | CTATATTATATCAACATCTA |
| 18 | P. ovale | coxIII | TATACCTTCATTATATAAAG |
| 19 | P. ovale | mitochondrial | CTTTCATATTAGTCATATTAT |
| | | | CTACAGCTG |
| 20 | P. ovale | mitochondrial | CCATTATAGGATTATTTACA |
| | | | ACAGTAAGTG |
| 21 | P. malariae | cytB | TAACTACTATTATACAATTC |
| 22 | P. malariae | coxI | GATTAACATTAGGTATATTA |
| 23 | P. malariae | coxIII | CCATCATTAATATAATATTC |
| 24 | P. malariae | mitochondrial | CATTAAGTACTTCTCTTATG |
| | | | TCTTTATCTC |
| 25 | P. malariae | mitochondrial | CTATGAGTTGTATAGCTATA |
| | | | TTAGGAAG |
| 26 | Plasmodium | mitochondrial | GGATAATTCTATTTATTAGG |
| | spp | | AGTCTC |
| 27 | Plasmodium | mitochondrial | AACAGGTTATAGTATAT |
| | spp | | ATAGAGCTC |
| 28 | Homo sapiens | mitochondrial | GCCAACTAATATTTCACTT |
| | | | TACATCCAAA |
| 29 | Homo sapiens | mitochondrial | GGCATTTTGTAGATGTGAT |
| | | | TTGACTATT |
| 74 | Homo sapiens | cytB | CATTATTGCAGCCCTAGCAA |
| 75 | Homo sapiens | coxI | ATACCTATTATTCGGCGCAT |
| 76 | Homo sapiens | coxIII | TTCCTCACTATCTGCTTCAT |

In an embodiment, the 5 different Plasmodium species in which the sensor is able to detect are Plasmodium falciparum, Plasmodium vivax, Plasmodium malariae, Plasmodium ovale, Plasmodium knowlesi.

In an embodiment, the isolated/synthetic nucleic acid probe for detecting the presence of single nucleotide polymorphism is an isolated/synthetic nucleic acid probe for detecting the presence of glucose-6-phosphate dehydrogenase single nucleotide polymorphism.

In an embodiment, the isolated/synthetic nucleic acid probe for detecting the presence of single nucleotide polymorphism comprise at least a sequence 90% identical to the sequences of the following list, or mixtures thereof. Preferably 91% identical, 92% identical, 93% identical, 94% identical, 95% identical, 96% identical, 97% identical, 98% identical, 99% identical or identical.

| SEQ ID | | | | |
|---|---|---|---|---|
| No | | | | |
| 30 | rs1050828a | Homo sapiens | g6pd | CATAGCCCACGATGAAGGTG |
| 31 | rs1050828b | Homo sapiens | g6pd | CATAGCCCATGATGAAGGTG |
| 32 | rs1050829a | Homo sapiens | g6pd | GGAGGGCATTCATGTGGCTG |
| 33 | rs1050829b | Homo sapiens | g6pd | GGAGGGCATACATGTGGCTG |
| 34 | rs1050829c | Homo sapiens | g6pd | GGAGGGCATCCATGTGGCTG |
| 35 | rs137852328a | Homo sapiens | g6pd | ATGTTGTCCCGGTTCCAGAT |
| 36 | rs137852328b | Homo sapiens | g6pd | ATGTTGTCCAGGTTCCAGAT |
| 37 | rs137852328c | Homo sapiens | g6pd | ATGTTGTCCTGGTTCCAGAT |
| 38 | rs76723693a | Homo sapiens | g6pd | GGGTCGTCCAGGTACCCTTT |
| 39 | rs76723693b | Homo sapiens | g6pd | GGGTCGTCCGGGTACCCTTT |
| 40 | rs5030872a | Homo sapiens | g6pd | GACAGCCGGTCAGAGCTCTGC |
| 41 | rs5030872b | Homo sapiens | g6pd | GACAGCCGGACAGAGCTCTGC |
| 42 | rs5030868a | Homo sapiens | g6pd | AACAGGGAGGAGATGTGGTT |
| 43 | rs5030868b | Homo sapiens | g6pd | AACAGGGAGAAGATGTGGTT |
| 44 | SNP | P falciparum | crtS1 | TGTAATGAATAAAATTTTTG |
| 45 | SNP | P falciparum | crtR1 | TGTAATTGAAACAATTTTTG |
| 46 | SNP | P falciparum | crtS2 | TTAATTAGTGCCTTAATTGT |
| 47 | SNP | P falciparum | crtR2 | TTAATTAGTTCCTTAATTGT |
| 48 | SNP | P falciparum | crtS3 | CATTTTTAAAACAACGTAAG |
| 49 | SNP | P falciparum | crtR3 | CATTTTTAAAAGAACGTAAG |
| 50 | SNP | P falciparum | crtS4 | CCTTCTTTAACATTTGTGAT |
| 51 | SNP | P falciparum | crtR4 | CCTTCTTTAGCATTTGTGAT |
| 52 | SNP | P falciparum | crtS5 | CCAGCAATAGCAATTGCTTA |
| 53 | SNP | P falciparum | crtR5 | CCAGCAACAGCAATTGCTTA |
| 54 | SNP | P falciparum | crtS6 | GATGTTGTAAGAGAACCAAG |
| 55 | SNP | P falciparum | crtR6 | GATGTTGTAATAGAACCAAG |
| 56 | SNP | P falciparum | mdr1S1 | AGAACATGAATTTAGGTGAT |
| 57 | SNP | P falciparum | mdr1R1 | AGAACATGTTITTAGGTGAT |
| 58 | SNP | P falciparum | mdr1S2 | TAGGTTTATATATTTGGTCA |
| 59 | SNP | P falciparum | mdr1R2 | TAGGTTTATATATTTGGTCA |
| 60 | SNP | P falciparum | mdr1S3 | ATGGGGATTCAGTCAAAGCG |
| 61 | SNP | P falciparum | mdr1R3 | ATGGGGATTCTGTCAAAGCG |
| 62 | SNP | P falciparum | mdr1S4 | TTATTTATTAATAGTTTTGC |
| 63 | SNP | P falciparum | mdr1R4 | TTATTTATTGATAGTTTTGC |
| 64 | SNP | P falciparum | mdr1S5 | AACTTAAGAGATCTTAGAAA |
| 65 | SNP | P falciparum | mdr1R5 | AACTTAAGATATCTTAGAAA |
| 66 | SNP | P falciparum | dhfrS1 | TGGAAATGTAATTCCCTAGA |
| 67 | SNP | P falciparum | dhfrR1 | TGGAAATGTATTTCCCTAGA |
| 68 | SNP | P falciparum | dhfrS2 | AAATATTTTTGTGCAGTTAC |
| 69 | SNP | P falciparum | dhfrR2 | AAATATTTTCGTGCAGTTAC |
| 70 | SNP | P falciparum | dhfrS3 | GAAGAACAAGCTGGGAAAGC |
| 71 | SNP | P falciparum | dhfrR3 | GAAGAACAAACTGGGAAAGC |
| 72 | SNP | P falciparum | dhfrS4 | GTTTTATTATAGGAGGTTCC |
| 73 | SNP | P falciparum | dhfrR4 | GTTTTATTTTAGGAGGTTCC |

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

Another aspect of the present disclosure relates to a kit for diagnosing malaria using a biological sample from a subject comprising the sensor described in any of the previous claims.

Another aspect of the present disclosure relates a method for obtaining the sensor of the present disclosure, comprising the following steps:
obtaining a graphene field-effect transistor comprising a graphene monolayer;
functionalizing the graphene monolayer with a linker, wherein the linker is selected from the following list: 1-pyrenebutyric acid succinimidyl ester, (9-fluorenylmethoxycarbonyloxy)succinimide, acridine orange succinimidyl ester, or mixtures thereof;
immobilizing a plurality of amine terminated isolated/synthetic nucleic acid probes, wherein the plurality of amine terminated isolated/synthetic nucleic acid probes comprise:
   at least 3 different isolated/synthetic nucleic acid probes for identifying the presence of at least 3 different Plasmodium species in the biological sample;
   at least 1 isolated/synthetic nucleic acid probe for identifying the presence of at least 1 Plasmodium species that is resistant to at least 1 antimalaria drug;
   at least 1 isolated/synthetic nucleic acid probe for detecting the presence of at least 1 single nucleotide polymorphism in the subject that influences the malaria treatment response of the subject.

In an embodiment the method may further comprise the step of: cleaning a graphene field-effect transistor comprising a graphene monolayer; passivating a gold region of the graphene field-effect transistor.

In an embodiment, the antimalaria drug resistance is resistance to a drug selected following list: chloroquine, mefloquine, doxycycline, atovaquone, proguanil.

### BRIEF DESCRIPTION OF THE DRAWINGS

The following figures provide preferred embodiments for illustrating the disclosure and should not be seen as limiting the scope of invention.

FIG. 1a shows the results of the electrical characterization of a subset of 8624 sensors. FIG. 1b shows an alternative multiplex layout.

FIG. 2 shows the calibration curves corresponding to the 7 studied artificial DNA sequences, in order (left to right, top to bottom): P. falciparum, P. vivax, P. malariae, P. ovale, P. knowlesi, P. spp and H. sapiens.

FIG. 3 shows the sensor response using different commercial saliva samples.

FIG. 4 shows the sensor response using the extracted parasite DNA in buffer (left) and in diluted type A saliva (right).

FIG. 5 shows an embodiment of the preparation of the monolayer graphene-based sensor for a rapid diagnosis of malaria using a non-invasive biological sample, of the present disclosure.

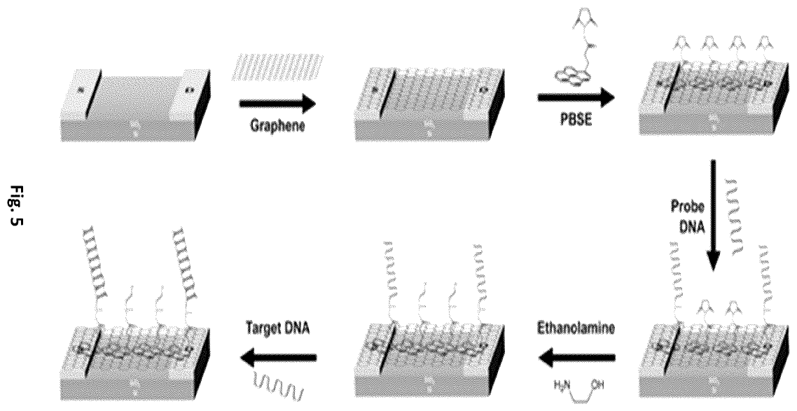

### DETAILED DESCRIPTION

The present disclosure relates to a monolayer graphene-based multiplex malaria diagnostic sensor. Specifically, a monolayer graphene-based sensor that is able to simultaneously detect the presence of different Plasmodium species, presence of drug-resistant Plasmodium species, and also the presence of G6PD single nucleotide polymorphisms in the test subject.

The present disclosure also relates to a monolayer graphene-based sensor, method, and kit for a rapid diagnosis of malaria using a non-invasive biological sample obtained from a subject, preferably in saliva or urine samples.

In an embodiment, the multiplex chip was obtained using a method comprising 7 lithography steps. The method was optimized to ensure that the chip comprise suitable full-coverage nitride passivation leaving open only the graphene sensor, similar to that described in previous works. In this optimization, a process for passivation of silicon nitride passivation of the graphene was developed, where a sacrificial nickel or copper thin film followed by aluminium is lithographically sputtered onto transferred silicon to protect graphene from the rest of the processes including deposition of the passivation, lithography, reactive ion etching, and wet etch of the sacrificial layer. The passivation covers source and drain electrodes, leaving only the graphene channel exposed. The process is described in P. D. Cabral et al, Clean-Room Lithographical Processes for the Fabrication of Graphene Biosensors. This passivation results in increased yield and uniformity of the sensor properties across the wafer.

In an embodiment, the method of obtaining the multiplex sensor comprises the following steps:
G-FETs cleaning with acetone rinsing (5 s) and immersion in ethyl acetate for 2 h. Rinsing with isopropyl alcohol (IPA) and DNAse, RNAse-free deionized water for 5 s each and dried under nitrogen flow;
Gold regions of the chip passivated with a fresh solution 20 μL of 2 mM 1-dodecanethiol (DDT) prepared in ethanol and incubated overnight (12 h) and rinsing with ethanol for 5 s and dried under nitrogen flow;
Graphene functionalization with 20 μL of 10 mM linker for 2 h at 20° C. and then rinsed for 5 s with the solvent used in this step and dry the chip under nitrogen flow;
Overnight immobilization of amine terminated synthetic nucleic acid probes (specific from malaria parasites) on the surface by adding 50-100 μL of 10 μM DNA probe prepared in phosphate buffer 10 mM (PB) pH 7.4 in DNAse, RNAse-free deionized water at 4° C. Surface rinsing for 5 s with PB and remove most of the solution without allowing full dryness;
Place 20 μL of 100 mM Ethanolamine prepared in PB pH 8.5 for 30 min and rinse it with PB for 5 s.

In an embodiment, each sensor or group of sensors is modified with suitable synthetic nucleic acid probes for multiplex detection. For tests with synthetic DNA target, 10 μL are placed on the suitable region of the chip. DNA target prepared in the 10 mM PB with 50 mM magnesium chloride and 150 mM sodium chloride pH 7, from the lowest to the highest concentration for 40 min each and rinse with PB for 5 s. In another embodiment, for real samples testing place 10 μL on the suitable region of the chip wait 40 min and rinse with PB for 5 s.

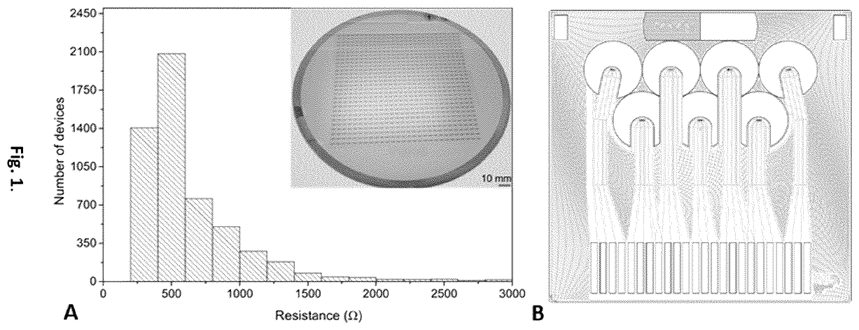

In an embodiment, the sensors obtained were characterized at the wafer level. It was observed that a large majority of the sensors exhibit good electrical properties, as measured by the zero-gate electrical channel resistance. FIG. 1a shows the results of the electrical characterization of a subset of 8624 sensors at the wafer level. In inset, picture of a 200 mm wafer with 784 chips each containing 20 sensors. The peak near 500Ω shows that a majority of the sensors have low resistance, a criterion for indicating the quality of the sensors obtained from the method of the present disclosure. An alternative multiplex layout is also shown FIG. 1b right.

In an embodiment, the sequences of the probes used to functionalize the sensors are selected from the following list:

| SEQ ID No | Function | | | |
|---|---|---|---|---|
| 1 | Malaria | P. falciparum | cytB | GTTTTAGTTATATTATCTAC |
| | Plasmodium | | | |
| 2 | Malaria | P. falciparum | coxI | ATATGCATATTATAGTATAC |
| | Plasmodium | | | |
| 3 | Malaria | P. falciparum | coxIII | CCTATAATCCTATTAATATT |
| | Plasmodium | | | |
| 6 | Malaria | P. vivax | cytB | GCTATATTAGTTAATACATA |
| | Plasmodium | | | |
| 7 | Malaria | P. vivax | coxI | CTATATTAATATCTATACCT |
| | Plasmodium | | | |
| 8 | Malaria | P. vivax | coxIII | CAATATAAGATATACCATAT |
| | Plasmodium | | | |
| 11 | Malaria | P. knowlesi | cytB | GTCATAACTAATTTATTATC |
| | Plasmodium | | | |
| 12 | Malaria | P. knowlesi | coxI | ATTCTATAATTATACTATGG |
| | Plasmodium | | | |
| 13 | Malaria | P. knowlesi | coxIII | GTATGAGGTAATAATATATA |
| | Plasmodium | | | |
| 16 | Malaria | P. ovale | cytB | TATACATATATTCTTCTTAC |
| | Plasmodium | | | |
| 17 | Malaria | P. ovale | coxI | CTATATTATATCAACATCTA |
| | Plasmodium | | | |
| 18 | Malaria | P. ovale | coxIII | TATACCTTCATTATATAAAG |
| | Plasmodium | | | |
| 21 | Malaria | P. malariae | cytB | TAACTACTATTATACAATTC |
| | Plasmodium | | | |
| 22 | Malaria | P. malariae | coxI | GATTAACATTAGGTATATTA |
| | Plasmodium | | | |
| 23 | Malaria | P. malariae | coxIII | CCATCATTAATATAATATTC |
| | Plasmodium | | | |
| 74 | control | Homo sapiens | cytB | CATTATTGCAGCCCTAGCAA |
| 75 | control | Homo sapiens | coxI | ATACCTATTATTCGGCGCAT |
| 76 | control | Homo sapiens | coxIII | TTCCTCACTATCTGCTTCAT |
| 30 | rs1050828a | Homo sapiens | g6pd | CATAGCCCACGATGAAGGTG |
| 31 | rs1050828b | Homo sapiens | g6pd | CATAGCCCATGATGAAGGTG |
| 32 | rs1050829a | Homo sapiens | g6pd | GGAGGGCATTCATGTGGCTG |
| 33 | rs1050829b | Homo sapiens | g6pd | GGAGGGCATACATGTGGCTG |
| 34 | rs1050829c | Homo sapiens | g6pd | GGAGGGCATCCATGTGGCTG |
| 35 | rs137852328a | Homo sapiens | g6pd | ATGTTGTCCCGGTTCCAGAT |
| 36 | rs137852328b | Homo sapiens | g6pd | ATGTTGTCCAGGTTCCAGAT |
| 37 | rs137852328c | Homo sapiens | g6pd | ATGTTGTCCTGGTTCCAGAT |
| 38 | rs76723693a | Homo sapiens | g6pd | GGGTCGTCCAGGTACCCTTT |
| 39 | rs76723693b | Homo sapiens | g6pd | GGGTCGTCCGGGTACCCTTT |
| 40 | rs5030872a | Homo sapiens | g6pd | GACAGCCGGTCAGAGCTCTGC |
| 41 | rs5030872b | Homo sapiens | g6pd | GACAGCCGGACAGAGCTCTGC |
| 42 | rs5030868a | Homo sapiens | g6pd | AACAGGGAGGAGATGTGGTT |
| 43 | rs5030868b | Homo sapiens | g6pd | AACAGGGAGAAGATGTGGTT |
| 44 | SNP | P falciparum | crtS1 | TGTAATGAATAAAATTTTTG |
| 45 | SNP | P falciparum | crtR1 | TGTAATTGAAACAATTTTTG |
| 46 | SNP | P falciparum | crtS2 | TTAATTAGTGCCTTAATTGT |
| 47 | SNP | P falciparum | crtR2 | TTAATTAGTTCCTTAATTGT |
| 48 | SNP | P falciparum | crtS3 | CATTTTTAAAACAACGTAAG |
| 49 | SNP | P falciparum | crtR3 | CATTTTTAAAAGAACGTAAG |
| 50 | SNP | P falciparum | crtS4 | CCTTCTTTAACATTTGTGAT |
| 51 | SNP | P falciparum | crtR4 | CCTTCTTTAGCATTTGTGAT |
| 52 | SNP | P falciparum | crtS5 | CCAGCAATAGCAATTGCTTA |
| 53 | SNP | P falciparum | crtR5 | CCAGCAACAGCAATTGCTTA |
| 54 | SNP | P falciparum | crtS6 | GATGTTGTAAGAGAACCAAG |
| 55 | SNP | P falciparum | crtR6 | GATGTTGTAATAGAACCAAG |
| 56 | SNP | P falciparum | mdr1S1 | AGAACATGAATTTAGGTGAT |
| 57 | SNP | P falciparum | mdr1R1 | AGAACATGTTTTTAGGTGAT |
| 58 | SNP | P falciparum | mdr1S2 | TAGGTTTATATATTTGGTCA |
| 59 | SNP | P falciparum | mdr1R2 | TAGGTTTATATATTTGGTCA |
| 60 | SNP | P falciparum | mdr1S3 | ATGGGGATTCAGTCAAAGCG |
| 61 | SNP | P falciparum | mdr1R3 | ATGGGGATTCTGTCAAAGCG |
| 62 | SNP | P falciparum | mdr1S4 | TTATTTATTAATAGTTTTGC |
| 63 | SNP | P falciparum | mdr1R4 | TTATTTATTGATAGTTTTGC |
| 64 | SNP | P falciparum | mdr1S5 | AACTTAAGAGATCTTAGAAA |
| 65 | SNP | P falciparum | mdr1R5 | AACTTAAGATATCTTAGAAA |
| 66 | SNP | P falciparum | dhfrS1 | TGGAAATGTAATTCCCTAGA |
| 67 | SNP | P falciparum | dhfrR1 | TGGAAATGTATTTCCCTAGA |
| 68 | SNP | P falciparum | dhfrS2 | AAATATTTTTGTGCAGTTAC |
| 69 | SNP | P falciparum | dhfrR2 | AAATATTTTCGTGCAGTTAC |
| 70 | SNP | P falciparum | dhfrS3 | GAAGAACAAGCTGGGAAAGC |
| 71 | SNP | P falciparum | dhfrR3 | GAAGAACAAACTGGGAAAGC |
| 72 | SNP | P falciparum | dhfrS4 | GTTTTATTATAGGAGGTTCC |
| 73 | SNP | P falciparum | dhfrR4 | GTTTTATTTTAGGAGGTTCC |
| 28 | control | Homo sapiens | Homo1 | GCCAACTAATATTTCACTTTAC |
| | | | | ATCCAAA |
| 29 | control | Homo sapiens | Homo2 | GGCATTTTGTAGATGTGATTT |
| | | | | GACTATT |
| 27 | Malaria | Plasmodium | Plasmodium | AACAGGTTATAGTATATATAG |
| | Plasmodium | spp | spp2 | AGCTC |
| 4 | Malaria | P. Falciparum | P. | GAACTCTATAAATAACCAGAC |
| | Plasmodium | | Falciparum | TATTTCAAC |
| | | | 1 | |
| 5 | Malaria | P. Falciparum | P. | CTGTAATTACTAACTTGTTATC |
| | Plasmodium | | Falciparum | CTCTATTC |
| | | | 2 | |
| 9 | Malaria | P. Vivax | P. Vivax | GTATGGATCGAATCTTACTTAT |
| | Plasmodium | | 1 | TCATATC |
| 10 | Malaria | P. Vivax | P. Vivax | TITAGTATCTGGTATTGCTAGT |
| | Plasmodium | | 2 | ATTATGTC |
| 24 | Malaria | P. Malariae | P. | CATTAAGTACTTCTCTTATGTC |
| | Plasmodium | | Malariae1 | TTTATCTC |
| 25 | Malaria | P. Malariae | P. | CTATGAGTTGTATAGCTATATT |
| | Plasmodium | | Malariae2 | AGGAAG |
| 26 | Malaria | Plasmodium | Plasmodium | GGATAATTCTATTTATTAGGAG |
| | Plasmodium | spp | spp1 | TCTC |
| 19 | Malaria | P. Ovale | P. Ovale1 | CTTTCATATTAGTCATATTATCT |
| | Plasmodium | | | ACAGCTG |
| 20 | Malaria | P. Ovale | P. Ovale2 | CCATTATAGGATTATTTACAAC |
| | Plasmodium | | | AGTAAGTG |
| 14 | Malaria | P. Knowlesi | P. | GAATATAATCACCTGTTATAAT |
| | Plasmodium | | Knowlesi1 | GTTCTAGG |
| 15 | Malaria | P. Knowlesi | P. | CCTTCACTATATAATGGATATG |
| | Plasmodium | | Knowlesi2 | GAGATAAA |

In an embodiment, the sensors obtained were characterized using spiked buffer.

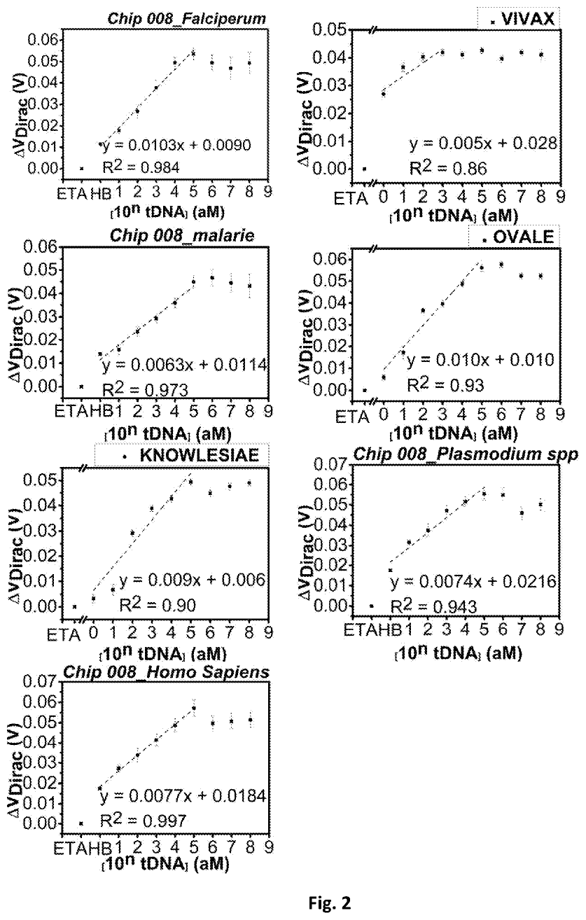

In an embodiment, the sensors were functionalized according to the procedure published in the paper by E. Fernandes et al. 2019 “Functionalization of single-layer graphene for immunoassays”. A sensor comprising 7 separate sensor groups for multiplex diagnosis was functionalized with 7 distinct deoxyribonucleic acid (DNA) probes. Each of the sensor groups was then calibrated with increasing concentrations of the corresponding DNA perfect match diluted in phosphate buffer (PB). FIG. 2 shows the calibration curves corresponding to the 7 different artificial DNA sequences: P. falciparum, P. vivax, P. malariae, P. ovale, P. knowlesi, P. spp and H. sapiens. All the sensor groups showed detection levels in the attomolar range.

The sensors showed consistent response starting in the attomolar range. FIG. 2 shows calibration data for the 7 probes selected, 5 probes specific to the malaria species, one common to all malaria, and one corresponding to humans. The sensors showed a sensitivity in the range of 6-10 mV/decade and a saturation signal in the range 30-50 mV.

In an embodiment, sensors that were functionalized with DNA and locked nucleic acid (LNA) probes showed similar responses as sensors functionalized with only DNA.

In an embodiment, the effectiveness of the functionalized sensors was tested using saliva and artificial DNA.

In an embodiment, the effectiveness of the functionalized sensors against complex matrices such as saliva were tested by using commercial saliva samples spiked with 1 μM of synthetic DNA sequence of Plasmodium falciparum fully complementary to sequence immobilized on the graphene surface.

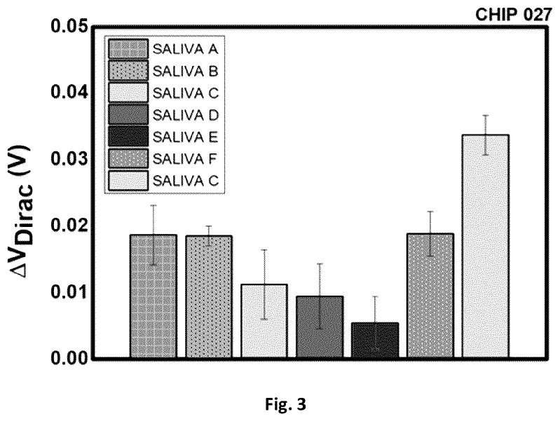

In an embodiment, the results show that the different saliva tested all show the same tendency, with shifts in signal enabling detection. Results were similar when the test was conducted using saliva samples collected from test individuals and pre-treated with an extraction kit or charcoal stripped. FIG. 3 shows the sensors' response for different commercial saliva samples spiked with 1 μM of target DNA for Plasmodium falciparum. All saliva samples tested yield a shift of electrical signal which indicates a positive test. Saliva A—adult 21-30 years old, saliva B—child 7-9 years old, saliva C—adult 31-40 years old, sample D—adult 21-30 years old, sample extracted with ThermoFisher brand kit, sample E—pooled (mixed) saliva, sample F—adult sample with charcoal stripped.

The results show that the different saliva samples collected from individuals in different age groups exhibit marked differences in signal level as compared to saliva samples from commercial providers corresponding to different age groups (3-10, adult).

In an embodiment, quantification of protein contents, ssDNA and dsDNA was performed for each saliva sample type. There was no clear correlation between saliva sample type and level of signal obtained.

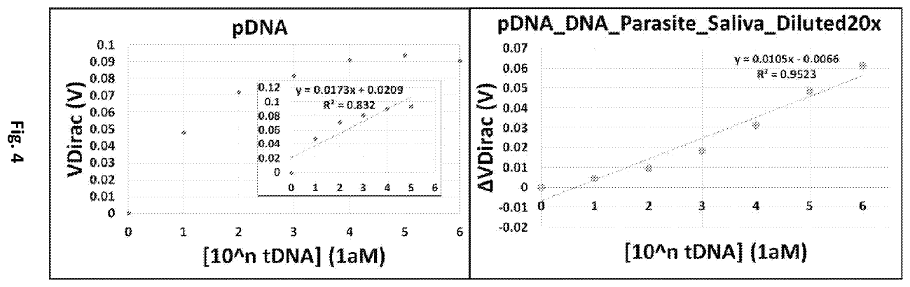

In an embodiment, the effectiveness of the functionalized sensors was further tested using saliva and natural DNA extracted from parasite culture. Parasites P. falciparum subtype Dd2 were cultured and its DNA was extracted using molecular biology techniques. A solution containing 2000 copies/μL of parasites DNA was used for testing. Sequential dilutions were performed to obtain concentrations in the range of 1 aM to 1 μM. The sensors were previously functionalized with a synthetic DNA probe for P. falciparum parasite. The extracted parasite DNA was mixed with PB, saliva or saliva diluted 20× with PB. The results of the test were shown in FIG. 4. The results show that the sensors were able to detect the parasite DNA dilutions and are able to detect as low as 1 aM concentration of parasite DNA in phosphate buffer and in diluted saliva samples. The samples of pure saliva (not shown) did not show consistent sensing behaviour, which we attribute to a difficulty, in the case of this experiment, to spread a viscous saliva sample onto the sensor, a problem which was solved by the dilution in buffer.

In an embodiment, the shelf-life and heat resistance capacity of the functionalized sensors were determined.

In an embodiment, the functionalized sensors were placed in the following conditions: 20° C., 45° C. at 75% relative humidity, 65° C. dry, 65° C. at 75% relative humidity. Thereafter, the sensors were tested after 1 week and after 2 weeks.

The sensors functionalized with DNA and LNA were shown to be working after heat treatment, often with improved effectiveness.

TABLE 1
Summarized sensor response for sensors functionalized with DNA
and LNA probes after different heat and humidity treatments.

| | DNA | DNA | LNA | LNA |
|---|---|---|---|---|
| | sensi- | total | sensi- | total |
| | tivity | shift | tivity | shift |
| Treatment | mV/dec | mV | mV/dec | mV |
| 20° C. dry 1 week | 11 | 80 | 6 | 50 |
| 20° C. dry 1 week | 9 | 350 | 53 | 350 |
| 45° C. 75% RH 1 week | 7 | 25 | 120 | 200 |
| 45° C. 75% RH 2 week | 6 | 30 | 68 | 250 |
| 65° C. dry 1 week | 43 | 180 | 22 | 180 |
| 65° C. dry 2 weeks | 34 | 170 | 25 | 180 |
| 45° C. 75% RH 1 week + | 52 | 200 | 14 | 80 |
| 65° C. 75% RH 1 week | | | | |

### Example 1

Each sensing region of the multiplex chips was functionalized overnight at 4° C. with 10 μL of specific probes for the different Plasmodium species, drug-resistant Plasmodium species, and G6PD single nucleotide polymorphism.

Each sensing region was rinsed for 5 sec with PB and most of the solution was removed without allowing full dryness. Then, 20 μL of 100 mM Ethanolamine prepared in PB pH 8.5 were placed on the chip for 30 min and rinsed with PB for 5 s. The chips were ready to use for sample analysis.

For the analysis, 10 μL of the saliva patient were added to each sensing region of the multiplex chips for 40 min, followed by PB rinsing for 5 s. If necessary, saliva can be diluted 20-fold in buffer.

The following cases might follow:
Results are negative for the tested parameters: no necessary treatment;
Results are positive only for non-resistance P. falciparum: treatment can be simpler medication instead of more radical treatments with artemisinin-based combination therapy due to resistance assumptions;
Results are positive for resistant P. falciparum: treatment according to World Health Organization recommendations;
Results are positive only for P. vivax: treatment can be chloroquine or pyrimethamine or sulfadoxine-pyrimethamine; instead, if positive to resistance—P. vivax—other drugs need to be used according to World Health Organization recommendations.
Results are positive only P. malariae: treatment according to World Health Organization recommendations;
Results are positive only P. knowlesi: treatment according to World Health Organization recommendations.

If the results are positive for a combination of multiple Plasmodium species with and without drug-resistance sensitivity, the drugs are immediately adjusted to the patient condition.

Independently of the type of infection, if patients are positive for G6PD gene the patient cannot be treated with Primaquine and Tafenoquineis due to the adverse effects (hemolysis) and possible death.

Currently, the rapid diagnosis of multiple infections is possible, however infections resistance and host mutation (G6PD single nucleotide polymorphism) assessment require laboratory equipment which take at least 24 h to provide results.

The present disclosure determines the diagnosis of multiple infectious with additional information of drug-resistance and G6PD single nucleotide polymorphism through a non-invasive saliva sample within less than 40 min. This detailed information assists the medical teams on suitable treatments increasing treatment success rates.

The disclosure should not be seen in any way restricted to the embodiments described and a person with ordinary skill in the art will foresee many possibilities to modifications thereof.

The embodiments described above are combinable.

This disclosure was funded by the Project MULTIMAL, ATTRACT ID 1176, funded by European Union's Horizon 2020 research and innovation programme under grant agreement No. 777222.

1-19. (canceled)

20. A monolayer graphene-based sensor for a rapid diagnosis of malaria using a non-invasive biological sample obtained from a subject, comprising:
at least 3 different isolated/synthetic nucleic acid probes for identifying the presence of at least 3 different Plasmodium species in the biological sample;
a linker for binding the at least 3 different isolated/synthetic nucleic acid probes to the graphene-based sensor, wherein the linker is selected from the group consisting of: 1-pyrenebutyric acid succinimidyl ester, (9-fluorenylmethoxycarbonyloxy)succinimide, acridine orange succinimidyl ester, and mixtures thereof;
at least 1 isolated/synthetic nucleic acid probe for identifying the presence of at least 1 Plasmodium species that is resistant to at least 1 antimalaria drug;
at least 1 isolated/synthetic nucleic acid probe for detecting the presence of at least 1 single nucleotide polymorphism in the subject that influence the malaria treatment response of the subject.

21. The sensor according to claim 20, wherein the non-invasive biological sample is a saliva sample or a urine sample.

22. The sensor according to claim 20, wherein the diagnosis of malaria takes less than one hour.

23. The sensor according to claim 20, wherein the sensor further comprises at least 1 isolated/synthetic nucleic acid probe for confirming the human origin of the biological sample.

24. The sensor according to claim 20, wherein the isolated/synthetic nucleic acid probes are selected from the group consisting of: deoxyribonucleic acid probes, ribonucleic acid probes, locked nucleic acid probes, and mixtures thereof.

25. The sensor according to claim 20, wherein the at least 3 different synthetic nucleic acid probes for identifying the presence of at least 3 different Plasmodium species in the biological sample comprise at least a sequence 90% identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

26. The sensor according to claim 20, wherein the at least 3 different synthetic nucleic acid probes for identifying the presence of at least 3 different Plasmodium species in the biological sample comprise at least a sequence 95% identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

27. The sensor according to claim 20, wherein the at least 3 different synthetic nucleic acid probes for identifying the presence of at least 3 different Plasmodium species in the biological sample comprise at least a sequence identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

28. The sensor according to claim 20, comprising at least 5 different synthetic nucleic acid probes for identifying the presence of at least 5 different Plasmodium species in the biological sample, wherein the at least 5 different nucleic acid probes comprise at least a sequence 90% identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

29. The sensor according to claim 20, comprising at least 5 different isolated/synthetic nucleic acid probes for identifying the presence of at least 5 different Plasmodium species in the biological sample, wherein the at least 5 different nucleic acid probes comprise at least a sequence 95% identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

30. The sensor according to claim 20, comprising at least 5 different isolated/synthetic nucleic acid probes for identifying the presence of at least 5 different Plasmodium species in the biological sample, wherein the 5 different nucleic acid probes comprise at least a sequence identical to the sequences selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

31. The sensor according to claim 30, wherein the at least 5 different Plasmodium species are Plasmodium falciparum, Plasmodium vivax, Plasmodium malariae, Plasmodium ovale, and Plasmodium knowlesi.

32. The sensor according to claim 20, wherein the at least 1 isolated/synthetic nucleic acid probe for detecting the presence of at least 1 single nucleotide polymorphism is an isolated/synthetic nucleic acid probe for detecting the presence of glucose-6-phosphate dehydrogenase single nucleotide polymorphism.

33. The sensor according to claim 20, wherein the at least 1 isolated/synthetic nucleic acid probe for detecting the presence of at least 1 single nucleotide polymorphism comprises at least a sequence 90% identical to the sequences selected from the group consisting of: SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76.

34. The sensor according to claim 20, wherein the isolated/synthetic nucleic acid probe for detecting the presence of single nucleotide polymorphism comprises at least a sequence 95% identical to the sequences selected from the group consisting of: SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76.

35. The sensor according to claim 20, wherein the isolated/synthetic nucleic acid probe for detecting the presence of single nucleotide polymorphism comprises at least a sequence identical to the sequences selected from the group consisting of: SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76.

36. The sensor according to claim 20, wherein the antimalaria drug resistance is resistant to a drug selected from the group consisting of: artemisinin, amodiaquine, chloroquine, mefloquine, doxycycline, atovaquone, and antifolates.

37. A kit for the diagnosing malaria using a biological sample from a subject comprising the sensor described in claim 20, comprising
at least 3 different isolated/synthetic nucleic acid probes for identifying the presence of at least 3 different Plasmodium species in the biological sample;
at least 1 isolated/synthetic nucleic acid probe for identifying the presence of at least 1 Plasmodium species that is resistant to at least 1 antimalaria drug; and
at least 1 isolated/synthetic nucleic acid probe for detecting the presence of at least 1 single nucleotide polymorphism in the subject that influences the malaria treatment response of the subject.

38. A method for obtaining the sensor according to claim 20 comprising the following steps:
obtaining a graphene field-effect transistor comprising a graphene monolayer;
functionalizing the graphene monolayer with a linker, wherein the linker is selected from the group consisting of: 1-pyrenebutyric acid succinimidyl ester, (9-fluorenylmethoxycarbonyloxy)succinimide, acridine orange succinimidyl ester, and mixtures thereof;
immobilizing a plurality of amine terminated isolated/synthetic nucleic acid probes, wherein the plurality of amine terminated isolated/synthetic nucleic acid probes comprise:
at least 3 different isolated/synthetic nucleic acid probes for identifying the presence of at least 3 different Plasmodium species in the biological sample;
at least 1 isolated/synthetic nucleic acid probe for identifying the presence of at least 1 Plasmodium species that is resistant to at least 1 antimalaria drug; and
at least 1 isolated/synthetic nucleic acid probe for detecting the presence of at least 1 single nucleotide polymorphism in the subject that influence the malaria treatment response of the subject.


SEQUENCE LISTING (76 sequences)

SEQ ID NO: 1 - DNA, 20 bp, Plasmodium falciparum
  cytB
gttttagttatattatctac

SEQ ID NO: 2 - DNA, 20 bp, Plasmodium falciparum
  coxI
atatgcatattatagtatac

SEQ ID NO: 3 - DNA, 20 bp, Plasmodium falciparum
  coxIII
cctataatcctattaatatt

SEQ ID NO: 4 - DNA, 30 bp, Plasmodium falciparum
  mitochondrial
gaactctataaataaccagactatttcaac

SEQ ID NO: 5 - DNA, 30 bp, Plasmodium falciparum
  mitochondrial
ctgtaattactaacttgttatcctctattc

SEQ ID NO: 6 - DNA, 20 bp, Plasmodium vivax
  cytB
gctatattagttaatacata

SEQ ID NO: 7 - DNA, 20 bp, Plasmodium vivax
  coxI
ctatattaatatctatacct

SEQ ID NO: 8 - DNA, 20 bp, Plasmodium vivax
  coxIII
caatataagatataccatat

SEQ ID NO: 9 - DNA, 29 bp, Plasmodium vivax
  mitochondrial
gtatggatcgaatcttacttattcatatc

SEQ ID NO: 10 - DNA, 30 bp, Plasmodium vivax
  mitochondrial
tttagtatctggtattgctagtattatgtc

SEQ ID NO: 11 - DNA, 20 bp, Plasmodium knowlesi
  cytB
gtcataactaatttattatc

SEQ ID NO: 12 - DNA, 20 bp, Plasmodium knowlesi
  coxI
attctataattatactatgg

SEQ ID NO: 13 - DNA, 20 bp, Plasmodium knowlesi
  coxIII
gtatgaggtaataatatata

SEQ ID NO: 14 - DNA, 30 bp, Plasmodium knowlesi
  mitochondrial
gaatataatcacctgttataatgttctagg

SEQ ID NO: 15 - DNA, 30 bp, Plasmodium knowlesi
  mitochondrial
ccttcactatataatggatatggagataaa

SEQ ID NO: 16 - DNA, 20 bp, Plasmodium ovale
  cytB
tatacatatattcttcttac

SEQ ID NO: 17 - DNA, 20 bp, Plasmodium ovale
  coxI
ctatattatatcaacatcta

SEQ ID NO: 18 - DNA, 20 bp, Plasmodium ovale
  coxIII
tataccttcattatataaag

SEQ ID NO: 19 - DNA, 30 bp, Plasmodium ovale
  mitochondrial
ctttcatattagtcatattatctacagctg

SEQ ID NO: 20 - DNA, 30 bp, Plasmodium ovale
  mitochondrial
ccattataggattatttacaacagtaagtg

SEQ ID NO: 21 - DNA, 20 bp, Plasmodium malariae
  cytB
taactactattatacaattc

SEQ ID NO: 22 - DNA, 20 bp, Plasmodium malariae
  coxI
gattaacattaggtatatta

SEQ ID NO: 23 - DNA, 20 bp, Plasmodium malariae
  coxIII
ccatcattaatataatattc

SEQ ID NO: 24 - DNA, 30 bp, Plasmodium malariae
  mitochondrial
cattaagtacttctcttatgtctttatctc

SEQ ID NO: 25 - DNA, 28 bp, Plasmodium malariae
  mitochondrial
ctatgagttgtatagctatattaggaag

SEQ ID NO: 26 - DNA, 26 bp, Plasmodium sp.
  mitochondrial
ggataattctatttattaggagtctc

SEQ ID NO: 27 - DNA, 26 bp, Plasmodium sp.
  mitochondrial
aacaggttatagtatatatagagctc

SEQ ID NO: 28 - DNA, 29 bp, Homo sapiens
  mitochondrial
gccaactaatatttcactttacatccaaa

SEQ ID NO: 29 - DNA, 28 bp, Homo sapiens
  mitochondrial
ggcattttgtagatgtgatttgactatt

SEQ ID NO: 30 - DNA, 20 bp, Homo sapiens
  rs5030872a g6pd
catagcccacgatgaaggtg

SEQ ID NO: 31 - DNA, 20 bp, Homo sapiens
  rs5030872b g6pd
catagcccatgatgaaggtg

SEQ ID NO: 32 - DNA, 20 bp, Homo sapiens
  rs5030868a g6pd
ggagggcattcatgtggctg

SEQ ID NO: 33 - DNA, 20 bp, Homo sapiens
  rs5030868b g6pd
ggagggcatacatgtggctg

SEQ ID NO: 34 - DNA, 20 bp, Homo sapiens
  SNP g6pd
ggagggcatccatgtggctg

SEQ ID NO: 35 - DNA, 20 bp, Homo sapiens
  SNP g6pd
atgttgtcccggttccagat

SEQ ID NO: 36 - DNA, 20 bp, Homo sapiens
  SNP g6pd
atgttgtccaggttccagat

SEQ ID NO: 37 - DNA, 20 bp, Homo sapiens
  SNP g6pd
atgttgtcctggttccagat

SEQ ID NO: 38 - DNA, 20 bp, Homo sapiens
  SNP g6pd
gggtcgtccaggtacccttt

SEQ ID NO: 39 - DNA, 20 bp, Homo sapiens
  SNP g6pd
gggtcgtccgggtacccttt

SEQ ID NO: 40 - DNA, 21 bp, Homo sapiens
  SNP g6pd
gacagccggtcagagctctgc

SEQ ID NO: 41 - DNA, 21 bp, Homo sapiens
  SNP g6pd
gacagccggacagagctctgc

SEQ ID NO: 42 - DNA, 20 bp, Homo sapiens
  SNP g6pd
aacagggaggagatgtggtt

SEQ ID NO: 43 - DNA, 20 bp, Homo sapiens
  SNP g6pd
aacagggagaagatgtggtt

SEQ ID NO: 44 - DNA, 20 bp, Plasmodium falciparum
  SNP crtS1
tgtaatgaataaaatttttg

SEQ ID NO: 45 - DNA, 20 bp, Plasmodium falciparum
  SNP crtR1
tgtaattgaaacaatttttg

SEQ ID NO: 46 - DNA, 20 bp, Plasmodium falciparum
  SNP crtS2
ttaattagtgccttaattgt

SEQ ID NO: 47 - DNA, 20 bp, Plasmodium falciparum
  SNP crtR2
ttaattagttccttaattgt

SEQ ID NO: 48 - DNA, 20 bp, Plasmodium falciparum
  SNP crtS3
catttttaaaacaacgtaag

SEQ ID NO: 49 - DNA, 20 bp, Plasmodium falciparum
  SNP crtR3
catttttaaaagaacgtaag

SEQ ID NO: 50 - DNA, 20 bp, Plasmodium falciparum
  SNP crtS4
ccttctttaacatttgtgat

SEQ ID NO: 51 - DNA, 20 bp, Plasmodium falciparum
  SNP crtR4
ccttctttagcatttgtgat

SEQ ID NO: 52 - DNA, 20 bp, Plasmodium falciparum
  SNP crtS5
ccagcaatagcaattgctta

SEQ ID NO: 53 - DNA, 20 bp, Plasmodium falciparum
  SNP crtR5
ccagcaacagcaattgctta

SEQ ID NO: 54 - DNA, 20 bp, Plasmodium falciparum
  SNP crtS6
gatgttgtaagagaaccaag

SEQ ID NO: 55 - DNA, 20 bp, Plasmodium falciparum
  SNP crtR6
gatgttgtaatagaaccaag

SEQ ID NO: 56 - DNA, 20 bp, Plasmodium falciparum
  SNP mdr1S1
agaacatgaatttaggtgat

SEQ ID NO: 57 - DNA, 20 bp, Plasmodium falciparum
  SNP mdr1R1
agaacatgtttttaggtgat

SEQ ID NO: 58 - DNA, 20 bp, Plasmodium falciparum
  SNP mdr1S2
taggtttatatatttggtca

SEQ ID NO: 59 - DNA, 20 bp, Plasmodium falciparum
  SNP mdr1R2
taggtttatatatttggtca

SEQ ID NO: 60 - DNA, 20 bp, Plasmodium falciparum
  SNP mdr1S3
atggggattcagtcaaagcg

SEQ ID NO: 61 - DNA, 20 bp, Plasmodium falciparum
  SNP mdr1R3
atggggattctgtcaaagcg

SEQ ID NO: 62 - DNA, 20 bp, Plasmodium falciparum
  SNP mdr1S4
ttatttattaatagttttgc

SEQ ID NO: 63 - DNA, 20 bp, Plasmodium falciparum
  SNP mdr1R4
ttatttattgatagttttgc

SEQ ID NO: 64 - DNA, 20 bp, Plasmodium falciparum
  mdr1S5
aacttaagagatcttagaaa

SEQ ID NO: 65 - DNA, 20 bp, Plasmodium falciparum
  mdr1R5
aacttaagatatcttagaaa

SEQ ID NO: 66 - DNA, 20 bp, Plasmodium falciparum
  dhfrS1
tggaaatgtaattccctaga

SEQ ID NO: 67 - DNA, 20 bp, Plasmodium falciparum
  dhfrR1
tggaaatgtatttccctaga

SEQ ID NO: 68 - DNA, 20 bp, Plasmodium falciparum
  dhfrS2
aaatatttttgtgcagttac

SEQ ID NO: 69 - DNA, 20 bp, Plasmodium falciparum
  dhfrR2
aaatattttcgtgcagttac

SEQ ID NO: 70 - DNA, 20 bp, Plasmodium falciparum
  dhfrS3
gaagaacaagctgggaaagc

SEQ ID NO: 71 - DNA, 20 bp, Plasmodium falciparum
  dhfrR3
gaagaacaaactgggaaagc

SEQ ID NO: 72 - DNA, 20 bp, Plasmodium falciparum
  dhfrS4
gttttattataggaggttcc

SEQ ID NO: 73 - DNA, 20 bp, Plasmodium falciparum
  dhfrR4
gttttattttaggaggttcc

SEQ ID NO: 74 - DNA, 20 bp, Homo sapiens
  control cytB
cattattgcagccctagcaa

SEQ ID NO: 75 - DNA, 20 bp, Homo sapiens
  control coxI
atacctattattcggcgcat

SEQ ID NO: 76 - DNA, 20 bp, Homo sapiens
  control coxIII
ttcctcactatctgcttcat